(19) United States
(12) Patent Application Publication
(10) Pub. No.: US 2007/0014802 A1
(43) Pub. Date: Jan. 18, 2007

(54) Modified antibody fragments

(75) Inventors: Sam Philip Heywood, Bucks (GB); David Paul Humphreys, Berkshire (GB)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Celltech R & D Limited, Berkshire (GB)

(21) Appl. No.: 10/562,769

(22) PCT Filed: Jul. 1, 2004
(86) PCT No.: PCT/GB04/02871

§ 371 Date: Jun. 27, 2006

(30) Foreign Application Priority Data
Jul. 1, 2003 (GB) 0315457.2

(51) Int. Cl. A61K 39/395; C07K 16/30; C07H 21/04; C12P 21/06

(52) U.S. Cl. 424/155.1; 530/388.8; 435/69.1; 435/326; 435/320.1; 536/23.53

(57) ABSTRACT

The present invention relates to a new class of antibody fragments including antibody Fab and Fab′ fragments in which the heavy chain is not covalently bonded to the light chain and two or more effector molecules are attached to the fragment, of which at least one of said molecules is attached to a cysteine in the heavy or light chain constant region.

The present invention relates to improved antibody fragments and more specifically provides improved antibody fragments to which two or more effector molecules are attached and methods for their production.

The high specificity and affinity of antibody variable regions make them ideal diagnostic and therapeutic agents, particularly for modulating protein:protein interactions. Antibody fragments are proving to be versatile therapeutic agents, as seen by the recent success of products such as ReoPro®. The targeting function encoded in Fv, Fab, Fab′, F(ab)₂ and other antibody fragments can be used directly or can be conjugated to one or more effector molecules such as cytotoxic drugs, toxins or polymer molecules to increase efficacy. For example, since these fragments lack an Fc region they have a short circulating half-life in animals but this can be improved by conjugation to certain types of polymer such as polyethylene glycol (PEG). Increasing the size of the conjugated PEG has been shown to increase the circulating half-life from minutes to many hours and modification of a Fab′ with PEG ranging from 5 kDa to 100 kDa has been demonstrated (Chapman et al., 1999, Nature Biotechnology, 17, 780-783; Leong et al., 2001, Cytokine, 16, 106-119; Chapman, 2002, Advanced Drug Delivery Reviews, 54, 531-545). PEGylated antibody fragments such as CDP870 are currently undergoing clinical trials where the effect of the conjugated PEG is to bring the circulating half-life to acceptable levels for therapy.

Effector molecules may be attached to antibody fragments by a number of different methods, including through aldehyde sugars or more commonly through any available amino acid side-chain or terminal amino acid functional group located in the antibody fragment, for example any free amino, imino, thiol, hydroxyl or carboxyl group. The site of attachment of effector molecules can be either random or site specific.

Random attachment is often achieved through amino acids such as lysine and this results in effector molecules being attached at a number of sites throughout the antibody fragment depending on the position of the lysines. While this has been successful in some cases the exact location and number of effector molecules attached cannot be controlled and this can lead to loss of activity for example if too few are attached and/or loss of affinity if for example they interfere with the binding site (Chapman 2002 Advanced Drug Delivery Reviews, 54, 531-545). As a result, controlled site specific attachment of effector molecules is usually the method of choice.

Site specific attachment of effector molecules is most commonly achieved by attachment to cysteine residues since such residues are relatively uncommon in antibody fragments. Antibody hinges are popular regions for site specific attachment since these contain cysteine residues and are remote from other regions of the antibody likely to be involved in antigen binding. Suitable hinges either occur naturally in the fragment or may be created using recombinant DNA techniques (See for example U.S. Pat. No. 5,677,425; WO98/25971; Leong et al., 2001 Cytokine, 16, 106-119; Chapman et al., 1999 Nature Biotechnology, 17, 780-783). Alternatively site specific cysteines may be engineered into the antibody fragment for example to create surface exposed cysteine(s) (U.S. Pat. No. 5,219,996).

Where effector molecules are to be site specifically attached via a cysteine, the target thiol in the antibody fragment is often capped by a small fermentation related peptide product such as glutathione or deliberately capped by a chemical additive used during antibody fragment extraction and purification such as 5,5′-dithiobis (2-nitrobenzoic acid) (DTNB). These capping agents need to be removed to activate the target (hinge or surface) thiol. Antibody fragments have a native interchain disulphide bond between the heavy and light chain constant regions (C_{H}1 and C_{L}) that has generally been regarded as critical in maintaining the stability and binding properties of the antibody. As a result the activation of the target hinge or surface thiol must be carried out with some care such that the inter C_{L}:C_{H}1 disulphide remains intact. Hence ‘mild’ reducing conditions are conventionally used to remove the thiol capping agent prior to reaction with the effector molecule. This is usually achieved by using thiol based reductants such as β-mercaptoethanol (β-ME), β-mercaptoethylamine (β-MA) and dithiothreitol (DTT). However, each of these reductants is known to be able to react with and stay attached to the cysteine which it is meant to reduce (Begg and Speicher, 1999 Journal of Biomolecular techniques, 10, 17-20) thereby reducing the efficiency of effector molecule attachment. Hence, following reduction and reaction with effector molecules, a large proportion of the antibody fragments do not have any effector molecules attached and these have to be purified away from the antibody fragments that have the correct number of effector molecules attached. This poor efficiency of modification is clearly a disadvantage during the large-scale production of modified therapeutic antibody fragments where it is important that maximum production efficiency is achieved.

Antibody fragments in which the heavy and light chains are not covalently linked have been described by Humphreys et al., 1997, Journal of Immunological Methods, 209, 193-202; Rodrigues et al., 1993, The Journal of Immunology, 151, 6954-6961; European Patent EP968291. The present invention provides a new class of modified antibody fragments in which the heavy and light chains are not covalently linked. Despite the absence of any covalent linkage between the heavy and the light chain and the attachment of two or more effector molecules, the fragments of the present invention perform comparably with wild type fragments in a number of in vitro and in vivo tests. Suprisingly these novel fragments have the same affinity for antigen and similar in vivo and in vitro stability as wild type fragments. A particular advantage of the fragments of the invention lies in their ease of manufacture, and in particular, their efficiency of manufacture. The fragments thus provide a low cost alternative to currently available fragments having inter-chain covalent linkages.

Thus according to the present invention there is provided an antibody Fab or Fab′ fragment in which the heavy chain in the fragment is not covalently bonded to the light chain characterized in that two or more effector molecules are attached to the fragment and at least one of said molecules is attached to a cysteine in the light chain or the heavy chain constant region.

The antibody fragment of the present invention may be any heavy chain and light chain pair having a variable (V_{H}/V_{L}) and constant region (C_{H}/C_{L}). The heavy and/or light chain constant region may be extended at its C-terminal with one or more amino acids. Particular examples include Fab and Fab′ fragments.

The antibody fragment starting material for use in the present invention may be obtained from any whole antibody, especially a whole monoclonal antibody, using any suitable enzymatic cleavage and/or digestion techniques, for example by treatment with pepsin. Alternatively, or in addition the antibody starting material may be prepared by the use of recombinant DNA techniques involving the manipulation and re-expression of DNA encoding antibody variable and/or constant regions. Standard molecular biology techniques may be used to modify, add or delete amino acids or domains as desired. Any alterations to the variable or constant regions are still encompassed by the terms ‘variable’ and ‘constant’ regions as used herein.

The antibody fragment starting material may be obtained from any species including for example mouse, rat, rabbit, pig, hamster, camel, llama, goat or human. Parts of the antibody fragment may be obtained from more than one species for example the antibody fragments may be chimeric. In one example the constant regions are from one species and the variable regions from another. The antibody fragment starting material may also be modified. In one example the variable region of the antibody fragment has been created using recombinant DNA engineering techniques. Such engineered versions include those created for example from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The methods for creating and manufacturing these antibody fragments are well known in the art (see for example, Boss et al., U.S. Pat. No. 4,816,397; Cabilly et al., U.S. Pat. No. 6,331,415; Shrader et al., WO 92/02551; Ward et al., 1989, Nature, 341, 544; Orlandi et al., 1989, Proc. Natl. Acad. Sci. USA, 86, 3833; Riechmann et al., 1988, Nature, 322, 323; Bird et al, 1988, Science, 242, 423; Queen et al., U.S. Pat. No. 5,585,089; Adair, WO91/09967; Mountain and Adair, 1992, Biotechnol. Genet. Eng. Rev, 10, 1-142; Verma et al., 1998, Journal of Immunological Methods, 216, 165-181).

Fab′ fragments for use in the present invention are extended at the C-terminus of the heavy chain by one or more amino acids. Typically the Fab′ fragments for use in the present invention possess a native or a modified hinge region. The native hinge region is the hinge region normally associated with the C_{H}1 domain of the antibody molecule. A modified hinge region is any hinge that differs in length and/or composition from the native hinge region. Such hinges can include hinge regions from other species, such as human, mouse, rat, rabbit, pig, hamster, camel, llama or goat hinge regions. Other modified hinge regions may comprise a complete hinge region derived from an antibody of a different class or subclass from that of the C_{H}1 domain. Thus, for instance, a C_{H}1 domain of class γ1 may be attached to a hinge region of class γ4. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as alanine, or by converting suitably placed residues into cysteine residues. By such means the number of cysteine residues in the hinge region may be increased or decreased. In addition other characteristics of the hinge can be controlled, such as the distance of the hinge cysteine(s) from the light chain interchain cysteine, the distance between the cysteines of the hinge and the composition of other amino acids in the hinge that may affect properties of the hinge such as flexibility e.g. glycines may be incorporated into the hinge to increase rotational flexibility or prolines may be incorporated to reduce flexibility. Alternatively combinations of charged or hydrophobic residues may be incorporated into the hinge to confer multimerisation properties. Other modified hinge regions may be entirely synthetic and may be designed to possess desired properties such as length, composition and flexibility.

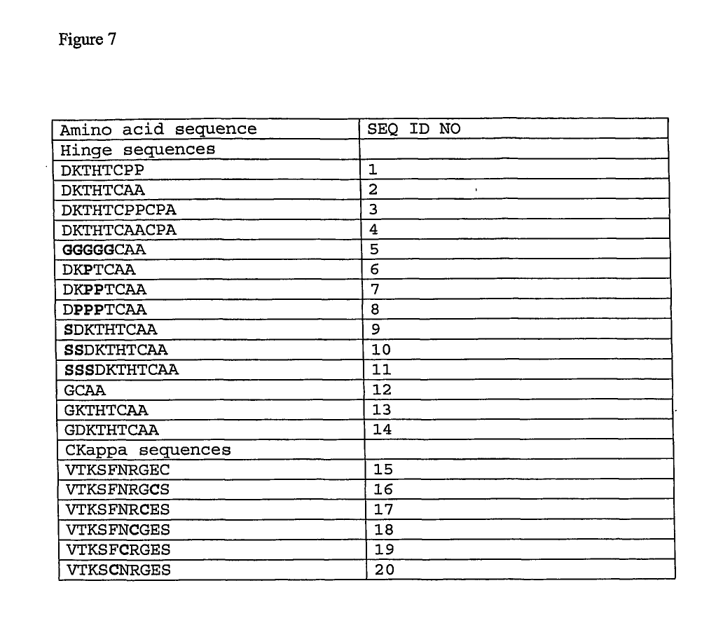

A number of modified hinge regions have already been described for example, in U.S. Pat. No. 5,677,425, WO9915549, and WO9825971 and these are incorporated herein by reference. Typically hinge regions for use in the present invention will contain between 1 and 11 cysteines. Preferably between 1 and 4 cysteines and more preferably 1 or 2 cysteines. Particularly useful hinges include a modified human γ1 hinge in which only one cysteine is present, comprising the sequence DKTHTCPP (SEQ ID NO:1) or DKTHTCAA (SEQ ID NO:2) and those containing two cysteines comprising the sequence DKTHTCPPCPA (SEQ ID NO:3) or DKTHTCAACPA (SEQ ID NO:4). Other suitable hinges for use in the present invention include those provided in SEQ ID NOs 5-11. Suitable murine hinge regions are provided in SEQ ID NOs 12-14. All sequences and their SEQ ID numbers are provided in FIG. 7.

The antibody fragment of the present invention will in general be capable of selectively binding to an antigen. The antigen may be any cell-associated antigen, for example a cell surface antigen on cells such as bacterial cells, yeast cells, T-cells, endothelial cells or tumour cells, or it may be a soluble antigen. Antigens may also be any medically relevant antigen such as those antigens upregulated during disease or infection, for example receptors and/or their corresponding ligands. Particular examples of cell surface antigens include adhesion molecules, for example integrins such as β1 integrins e.g. VLA-4, E-selectin, selectin or L-selectin, CD2, CD3, CD4, CD5, CD7, CD8, CD11a, CD11b, CD18, CD19, CD20, CD23, CD25, CD33, CD38, CD40, CD45, CDW52, CD69, carcinoembryonic antigen (CEA), human milk fat globulin (HMFG1 and 2), MHC Class I and MHC Class II antigens, and VEGF, and where appropriate, receptors thereof. Soluble antigens include interleukins such as IL-1, IL-2, IL-3, IL-4, IL-5, IL-6, IL-8, IL-12, IL-16 or IL-17, viral antigens for example respiratory syncytial virus or cytomegalovirus antigens, immunoglobulins, such as IgE, interferons such as interferon α, interferon β or interferon γ, tumour necrosis factor-α, tumor necrosis factor-β, colony stimulating factors such as G-CSF or GM-CSF, and platelet derived growth factors such as PDGF-α, and PDGF-β and where appropriate receptors thereof.

The term effector molecule as used herein includes, for example, antineoplastic agents, drugs, toxins (such as enzymatically active toxins of bacterial or plant origin and fragments thereof e.g. ricin and fragments thereof) biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Particular antineoplastic agents include cytotoxic and cytostatic agents for example alkylating agents, such as nitrogen mustards (e.g. chlorambucil, melphalan, mechlorethamine, cyclosphophamide, or uracil mustard) and derivatives thereof, triethylenephosphoramide, triethylenethiophosphor-amide, busulphan, or cisplatin; antimetabolites, such as methotrexate, fluorouracil, floxuridine, cytarabine, mercaptopurine, thioguanine, fluoroacetic acid, or fluorocitric acid, antibiotics, such as bleomycins (e.g. bleomycin sulphate), doxorubicin, daunorubicin, mitomycins (e.g. mitomycin C), actionmycins (e.g. dactinomycin) plicamyin, calichaemicin and derivatives thereof, or esperamicin and derivatives thereof; mitotic inhibitors, such as etoposide, vincristine or vinblastine and derivatives thereof; alkaloids such as ellipticine; polyols such as taxicin-I or taxicin-II; hormones, such as androgens (e.g. dromostanolone or testolactone), progestins (e.g. megestrol acetate or medroxyprogesterone acetate), estrogens (e.g. dimethylstilbestrol diphosphate, polyestradiol phosphate or estramustine phosphate) or antiestrogens (e.g. tamoxifen); anthraquinones, such as mitoxantrone, ureas, such as hydroxyurea; hydrazines, such as procarbazine; or imidazoles, such as dacarbazine.

Chelated metals include chelates of di- or tripositive metals having a coordination number from 2 to 8 inclusive. Particular examples of such metals include technetium (Tc), rhenium (Re), cobalt (Co), copper (Cu), gold (Au), silver (Ag), lead (Pb), bismuth (Bi), indium (In), gallium (Ga), yttrium (Y), terbium (Th), gadolinium (Gd), and scandium (Sc). In general the metal is preferably a radionuclide. Particular radionuclides include ^{99m}Tc, ¹⁸⁶Re, ¹⁸⁸Re, ⁵⁸Co, ⁶⁰Co, ⁶⁷Cu, ¹⁹⁵Au, ¹⁹⁹Au, ¹¹⁰Ag, ²⁰³Pb, ²⁰⁶Bi, ²⁰⁷Bi, ¹¹¹In, ⁶⁷Ga, ⁶⁸Ga, ⁸⁸Y, ⁹⁰Y, ¹⁶⁰Tb, ¹⁵³Gd and ⁴⁷Sc.

The chelated metal may be for example one of the above types of metal chelated with any suitable polyadentate chelating agent, for example acyclic or cyclic polyamines, polyethers, (e.g. crown ethers and derivatives thereof); polyamides; porphyrins; and carbocyclic derivatives.

In general, the type of chelating agent will depend on the metal in use. One particularly useful group of chelating agents in conjugates according to the invention, however, are acyclic and cyclic polyamines, especially polyaminocarboxylic acids, for example diethylenetriaminepentaacetic acid and derivatives thereof, and macrocyclic amines, e.g. cyclic tri-aza and tetra-aza derivatives (for example as described in International Patent Specification No. WO 92/22583); and polyamides, especially desferriox-amine and derivatives thereof.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

Synthetic or naturally occurring polymers for use as effector molecules include, for example optionally substituted straight or branched chain polyalkylene, polyalkenylene, or polyoxyalkylene polymers or branched or unbranched polysaccharides, e.g. a homo- or hetero-polysaccharide such as lactose, amylose, dextran or glycogen.

Particular optional substituents which may be present on the above-mentioned synthetic polymers include one or more hydroxy, methyl or methoxy groups. Particular examples of synthetic polymers include optionally substituted straight or branched chain poly(ethyleneglycol), poly(propyleneglycol), poly(vinylalcohol) or derivatives thereof, especially optionally substituted poly(ethyleneglycol) such as methoxypoly(ethyleneglycol) or derivatives thereof.

“Derivatives” as used herein is intended to include reactive derivatives, for example thiol-selective reactive groups such as an α-halocaraboxylic acid or ester, e.g. iodoacetamide, an imide, e.g. maleimide, a vinyl sulphone or disulphide malemides and the like. The reactive group may be linked directly or through a linker segment to the polymer. It will be appreciated that the residue of such a group will in some instances form part of the product as the linking group between the antibody fragment and the polymer. The size of the polymer may be varied as desired, but will generally be in an average molecular weight range from 500 Da to 50,000 Da, preferably from 5,000 to 40,000 Da and more preferably from 10,000 to 40,000 Da and 20,000 to 40,000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumor, it may be advantageous to use a small molecular weight polymer, for example with a molecular weight of around 5,000 Da. For applications where the product remains in the circulation, it may be advantageous to use a higher molecular weight polymer, for example having a molecular weight in the range from 25,000 Da to 40,000 Da.

Particularly preferred polymers include a polyalkylene polymer, such as a poly(ethyleneglycol) or, especially, a methoxypoly(ethyleneglycol) or a derivative thereof, and especially with a molecular weight in the range from about 10,000 Da to about 40,000 Da.

The polymers of the present invention may be obtained commercially (for example from Nippon Oil and Fats; Nektar Therapeutics) or may be prepared from commercially available starting materials using conventional chemical procedures.

Effector molecules of the present invention may be attached using standard chemical or recombinant DNA procedures in which the protein is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include for example those described in International Patent Specification numbers WO 93/06231, WO92/22583, WO90/09195, WO89/01476, WO9915549 and WO03031581. Alternatively, where the effector molecule is a protein or polypeptide the linkage may be achieved using recombinant DNA procedures, for example as described in European Patent Specification No. 392745.

In one example the effector molecules of the present invention may be attached to the protein through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods. See for example U.S. Pat. No. 5,219,996. In a preferred aspect of the invention an effector molecule is covalently linked through a thiol group of a cysteine residue located in the fragment. The covalent linkage will generally be a disulphide bond or, in particular, a sulphur-carbon bond. In one example where a thiol group is used as the point of attachment appropriately activated effector molecules, for example thiol selective derivatives such as maleimides and cysteine derivatives may be used.

In a preferred aspect of the present invention at least one of the effector molecules attached to the antibody fragment is a polymer molecule, preferably PEG or a derivative thereof. As regards attaching poly(ethyleneglycol) (PEG) moieties in general, reference is made to “Poly(ethyleneglycol) Chemistry, Biotechnical and Biomedical Applications”, 1992, J. Milton Harris (ed), Plenum Press, New York; “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York.

In one example of the present invention all the effector molecules are PEG and each molecule is covalently linked via a maleimide group to one or more thiol groups in the antibody fragment. The PEG may be any straight or branched molecule. To attach branched PEG molecules, a lysine residue is preferably covalently linked to the maleimide group. To each of the amine groups on the lysine residue is preferably attached a methoxy(poly(ethyleneglycol) polymer. In one example the molecular weight of each polymer is approximately 20,000 Da and the total molecular weight of the entire polymer molecule is therefore approximately 40,000 Da.

In the present invention two or more effector molecules are attached to the antibody fragment and at least one of said molecules is attached to a cysteine in the light chain or the heavy chain constant region. Suitable cysteines for attachment include naturally occurring cysteines present in the light and/or heavy chain constant region and cysteines that have been engineered into the constant regions using recombinant DNA techniques. In one example two cysteines are engineered into the antibody fragment, one in each of the heavy and light chain constant regions. In one particular example these cysteines are engineered at positions whereby they can form a disulphide linkage with each other in the antibody starting material.

In one example of the present invention at least one effector molecule is attached to an interchain cysteine. The term interchain cysteine as used herein refers to a cysteine in the heavy or light chain constant region that would be disulphide linked to a cysteine in the corresponding heavy or light chain constant region in a naturally occurring antibody molecule. In particular the interchain cysteines of the present invention are a cysteine in the constant region of the light chain (C_{L}) and a cysteine in the first constant region of the heavy chain (C_{H}1) that are disulphide linked to each other in naturally occurring antibodies. Examples of such cysteines may typically be found at position 214 of the light chain and 233 of the heavy chain of human IgG1, 127 of the heavy chain of human IgM, IgE, IgG2, IgG3, IgG4 and 128 of the heavy chain of human IgD and IgA2B, as defined by Kabat et al., 1987, in Sequences of Proteins of Immunological Interest, US Department of Health and Human Services, NIH, USA. In murine IgG1, interchain cysteines may be found at position 214 of the light chain and 235 of the heavy chain. It will be appreciated that the exact positions of these cysteines may vary from that of naturally occurring antibodies if any modifications, such as deletions, insertions and/or substitutions have been made to the antibody starting material. Hence according to one example of the present invention two or more effector molecules are attached to the antibody fragment and at least one of said molecules is attached to the interchain cysteine of C_{L} or the interchain cysteine of C_{H}1.

In the antibody fragments of the present invention, to which two or more effector molecules are attached, the heavy chain is not covalently bonded to the light chain. In these fragments there are no disulphide linkages between the heavy and the light chain and in particular the disulphide linkage found in naturally occurring antibodies between the interchain cysteine of C_{L} and the interchain cysteine of C_{H}1 is absent.

In one example of the present invention the covalent linkage between the two interchain cysteines is absent as a result of one of the interchain cysteines being replaced with another amino acid, preferably an amino acid that does not contain a thiol group. By replace we mean that where the interchain cysteine would normally be found in the antibody fragment another amino acid is in its place. Examples of suitable amino acids include serine, threonine, alanine, glycine or any polar amino acid. A particularly preferred amino acid is serine. The methods for replacing amino acids are well known in the art of molecular biology. Such methods include for example site directed mutagenesis using methods such as PCR to delete and/or substitute amino acids or de novo design of synthetic sequences. Fab′ and F(ab′)₂ in which both the interchain cysteines have been replaced by serines have already been described (Humphreys et al., 1997, Journal of Immunological Methods, 209, 193-202; Rodrigues et al., 1993, The Journal of Immunology, 151, 6954-6961). Hence according to one aspect of the present invention antibody Fab and Fab′ fragments are provided in which one of the interchain cysteines has been replaced by another amino acid, preferably an amino acid that does not contain a thiol group, even more preferably by serine. Particular fragments according this aspect of the invention are:
(i) An antibody Fab′ fragment characterized in that the interchain cysteine of C_{H}1 has been replaced by another amino acid.
(ii) An antibody Fab′ fragment characterized in that the interchain cysteine of C_{L} has been replaced by another amino acid.
(iii) An antibody Fab fragment characterized in that the interchain cysteine of C_{H}1 has been replaced by another amino acid.
(iv) An antibody Fab fragment characterized in that the interchain cysteine of C_{L} has been replaced by another amino acid.
   Two or more effector molecules may be attached to these fragments and according to one aspect of the present invention an effector molecule is attached to one of the interchain cysteines of C_{L} or C_{H}1 and additional effector molecules are attached elsewhere in the antibody fragment, in particular the constant region and/or the hinge region. Preferably additional effector molecules are attached to the hinge.
   Particular fragments according to this aspect of the invention are those where:
(i) an effector molecule is attached to the interchain cysteine of C_{L} and the interchain cysteine of C_{H}1 has been replaced by another amino acid or
(ii) an effector molecule is attached to the interchain cysteine of C_{H}1 and the interchain cysteine of C_{L} has been replaced by another amino acid
   In another example of the present invention an effector molecule is attached to at least one cysteine in the light chain constant region and at least one cysteine in the heavy chain constant region. As described above suitable cysteines include naturally occurring cysteines present in the light and/or heavy chain constant region, such as the interchain cysteines of C_{H}1 and C_{L} and cysteines that have been engineered into the constant regions using recombinant DNA techniques. In one particular example each cysteine to which an effector molecule is attached would otherwise be linked to a cysteine in the corresponding heavy or light chain via a disulphide bond if the effector molecules were not attached. In this example the covalent linkage between the two cysteines is removed during attachment of the effector molecules, as described herein, using a reducing agent. Additional effector molecules may be attached elsewhere in the antibody fragment, in particular the constant region and/or the hinge using any of the methods described herein. Preferably additional effector molecules are attached to the hinge.

Particular fragments according to this aspect of the invention include those where:
(i) the cysteine residues in the heavy and light chain constant regions which are attached to effector molecules would otherwise be linked to each other via a disulphide bond if the effector molecules were not attached or
(ii) the light chain cysteine to which an effector molecule is attached is the interchain cysteine of C_{L} and the heavy chain cysteine to which an effector molecule is attached is the interchain cysteine of C_{H}1

Also provided by the present invention are antibody Fab′ fragment intermediates that are useful in producing some of the antibody fragments of the present invention. Surprisingly it has been found that the interchain cysteine of C_{L} can form a disulphide linkage with a cysteine in the hinge region when the interchain cysteine of C_{H}1 has been substituted with a non-thiol containing amino acid. The presence of the disulphide linkage between the hinge cysteine and the C_{L} interchain cysteine allows the modified antibody Fab′ fragment to be purified as efficiently as Fab′ fragments containing a native interchain disulphide by enabling the Fab′ fragment to be extracted using heat extraction methods at 60° C. or greater (see U.S. Pat. No. 5,665,866). Hence according to this aspect of the invention there is provided an antibody Fab′ fragment, characterized in that the C_{H}1 interchain cysteine has been replaced by a non-thiol containing amino acid and the C_{L} interchain cysteine is covalently bonded to a cysteine in the hinge region. Any of the hinges previously described may be used in this intermediate but in particular the hinge region of said intermediate is of sufficient length and flexibility to enable a cysteine in said hinge to form a disulphide linkage with the interchain cysteine of C_{L}. Particularly useful hinges include a modified human γ1 hinge in which only one cysteine is present, comprising the sequence DKTHTCPP (SEQ ID NO:1) or DKTHTCAA (SEQ ID NO:2). Alternatively the hinge may contain two cysteines for example DKTHTCPPCPA (SEQ ID NO:3) or DKTHTCAACPA (SEQ ID NO:4). Additional hinges for use in these antibody fragments include those provided in SEQ ID NOs 5-11 and in murine constant regions, the sequences provided in SEQ ID NOs 12-14. In one example the light chain constant region in the antibody Fab′ fragment which contains the interchain cysteine to which the hinge cysteine is covalently bonded is ckappa from human IgG1 (SEQ ID NO:15).

Other useful intermediates which also contain a disulphide bond between the hinge and the light chain are antibody Fab′ fragments characterized in that the heavy chain in the fragment is not covalently bonded to the light chain, both the interchain cysteine of C_{H}1 and C_{L} have been replaced by another amino acid and an engineered cysteine in the light chain constant region is covalently bonded to a cysteine in the hinge region. The term ‘engineered cysteine’ refers to a cysteine at a position in the light chain constant region other than that of the interchain cysteine. The methods for replacing and inserting amino acids are well known in the art of molecular biology. Such methods include for example site directed mutagenesis using methods such as PCR to delete and/or substitute amino acids or de novo design of synthetic sequences. Particular light chain constant region sequences for use in this aspect of the present invention are provided in SEQ ID NOs 16-20. Particular hinge sequences that may be used with any of the light chain constant region sequences provided in SEQ ID NOs 16-20 are provided in SEQ ID NOs 1-11.

Two or more effector molecules may be attached to the antibody Fab′ fragments of this aspect of the invention. Hence according to one aspect of the present invention an effector molecule is attached to either the interchain cysteine of C_{L} or an engineered cysteine in the light chain constant region, whichever is present and additional effector molecules are attached elsewhere in the antibody fragment, in particular the hinge region. Preferably additional effector molecules are attached to the hinge.

Hence in one aspect an effector molecule is attached to a cysteine in the hinge which was covalently linked to the interchain cysteine of C_{L} prior to attachment of the effector molecules. In another aspect an effector molecule is attached to a cysteine in the hinge which was covalently linked to an engineered cysteine in the light chain constant region prior to attachment of the effector molecules.

Also provided by the present invention is a host cell expressing the antibody Fab′ fragment intermediate described above. Any suitable host cell/vector system may be used for the expression of the DNA sequences encoding the antibody Fab′ intermediate of the present invention. Bacterial, for example E. coli, and other microbial systems may be used or eukaryotic, for example mammalian host cell expression systems may also be used. Suitable E. coli strains for use in the present invention may be naturally occurring strains or mutated strains capable of producing recombinant proteins. Examples of specific host E. coli strains include MC4100, TG1, TG2, DHB4, DH5a, DH1, BL21, XL1Blue and W3110 (ATCC 27,325). Suitable mammalian host cells include CHO, myeloma or hybridoma cells.

Also provided by the present invention are methods for attaching effector molecules to the antibody Fab or Fab′ fragment(s) of the present invention. In general the methods comprise:
a) Treating an antibody Fab or Fab′ fragment with a reducing agent capable of generating a free thiol group in a cysteine of the heavy and/or light chain constant region
b) Reacting the treated fragment with an effector molecule
   In one aspect of the invention where the interchain disulphide bond is present in the antibody fragment prior to attachment of the effector molecules the method comprises:
a) Treating an antibody Fab or Fab′ fragment with a reducing agent capable of generating a free thiol group in at least the interchain cysteine of C_{H}1 and the interchain cysteine of C_{L}.
b) Reacting the treated fragment with an effector molecule
   In one aspect of the invention where one of the antibody Fab′ intermediates described above is used there is provided a method of attaching two or more effector molecules to the antibody Fab′ intermediate, said method comprising:
a) Treating an antibody Fab′ fragment with a reducing agent capable of reducing the covalent bond between the C_{L} interchain cysteine and a cysteine in the hinge region
b) Reacting the treated fragment with an effector molecule
   In another aspect where one of the antibody Fab′ intermediates described above is used there is provided a method of attaching two or more effector molecules to the antibody Fab′ intermediate, said method comprising:
a) Treating an antibody Fab′ fragment with a reducing agent capable of reducing the covalent bond between an engineered cysteine in the light chain constant region and a cysteine in the hinge region
b) Reacting the treated fragment with an effector molecule
   The methods provided by the present invention enable one or more effector molecule(s) to be attached to cysteines in the antibody fragment, in particular to cysteines in the constant region and the hinge. Two or more effector molecules can be attached to the antibody fragment using the methods described herein either simultaneously or sequentially by repeating the method.

The methods of the present invention also extend to one or more steps before and/or after the reduction methods described above in which further effector molecules are attached to the antibody fragment using any suitable method as described previously, for example via other available amino acid side chains such as amino and imino groups.

The reducing agent for use in the methods of the present invention is any reducing agent capable of reducing cysteines in the antibody fragment starting material to produce free thiols. Preferably the reducing agent efficiently reduces all available thiols. In one aspect of the present invention the reducing agent will need to be strong enough to reduce the interchain disulphide bond between cysteines of the heavy and light chain constant regions, for example, between the interchain cysteine of C_{L} and the interchain cysteine of C_{H}1, in order to allow attachment of effector molecules to said cysteines. Where the interchain disulphide bond is absent due to the absence of one of the interchain cysteines, the reducing agent must be capable of efficiently liberating free thiols from the remaining cysteine(s) in the antibody fragment e.g. the remaining interchain cysteine and/or a cysteine in the hinge region. As the antibody molecules of the present invention have no requirement for the interchain disulphide bond stronger reducing agents can be used than are conventionally used with wild type antibody fragments. As a result a higher number of free thiols are produced and a higher proportion of the antibody fragments are correctly modified i.e. the correct number of effector molecules are attached. The antibody fragments of the present invention can therefore be produced more efficiently and cost effectively than conventional antibody fragments. It will be clear to a person skilled in the art that suitable reducing agents may be identified by determining the number of free thiols produced after the antibody fragment is treated with the reducing agent. Methods for determining the number of free thiols are well known in the art, see for example Lyons et al., 1990, Protein Engineering, 3, 703. Reducing agents for use in the present invention are widely known in the art for example those described in Singh et al., 1995, Methods in Enzymology, 251, 167-73. Particular examples include thiol based reducing agents such as reduced glutathione (GSH), β-mercaptoethanol (β-ME), β-mercaptoethylamine (β-MA) and dithiothreitol (DTT). Other methods for reducing the antibody fragments of the present invention include using electrolytic methods, such as the method described in Leach et al., 1965, Div. Protein. Chem, 4, 23-27 and using photoreduction methods, such as the method described in Ellison et al., 2000, Biotechniques, 28 (2), 324-326. Preferably however, the reducing agent for use in the present invention is a non-thiol based reducing agent capable of liberating one or more thiols in an antibody fragment. Preferably the non-thiol based reducing agent is capable of liberating all available thiols in an antibody fragment. Preferred reducing agents for use in the present invention are trialkylphosphine reducing agents (Ruegg U T and Rudinger, J., 1977, Methods in Enzymology, 47, 111-126; Burns J et al., 1991, J. Org. Chem, 56, 2648-2650; Getz et al., 1999, Analytical Biochemistry, 273, 73-80; Han and Han, 1994, Analytical Biochemistry, 220, 5-10; Seitz et al., 1999, Euro. J. Nuclear Medicine, 26, 1265-1273). Particular examples of which include tris(2-carboxyethyl)phosphine (TCEP), tris butyl phosphine (TBP), tris-(2-cyanoethyl)phosphine, tris-(3-hydroxypropyl)phosphine (THP) and tris-(2-hydroxyethyl)phosphine. Most preferably the reducing agent for use in the present invention is either TCEP or THP. It will be clear to a person skilled in the art that the concentration of reducing agent for use in the present invention can be determined empirically, for example, by varying the concentration of reducing agent and measuring the number of free thiols produced. Typically the reducing agent for use in the present invention is used in excess over the antibody fragment for example between 2 and 1000 fold molar excess. Preferably the reducing agent is in 2, 3, 4, 5, 10, 100 or 1000 fold excess. In one preferred example the reducing agent is in 4 molar excess.

The modified antibody fragments according to the invention may be prepared by reacting an antibody fragment (as described herein) containing at least one reactive cysteine residue with an effector molecule, preferably a thiol-selective activated effector molecule. The reactions in steps (a) and (b) of the methods described above may generally be performed in a solvent, for example an aqueous buffer solution such as acetate or phosphate, at around neutral pH, for example around pH 4.5 to around pH 8.0. The reaction may generally be performed at any suitable temperature, for example between about 5° C. and about 70° C., for example at room temperature. The solvent may optionally contain a chelating agent such as EDTA, EGTA, CDTA or DTPA. Preferably the solvent contains EDTA at between 1 and 5 mM, preferably 2 mM. Alternatively or in addition the solvent may be a chelating buffer such as citric acid, oxalic acid, folic acid, bicine, tricine, tris or ADA. The effector molecule will generally be employed in excess, concentration relative to the concentration of the antibody fragment. Typically the effector molecule is in between 2 and 100 fold molar excess, preferably 5, 10 or 50 fold excess.

Where necessary, the desired product containing the desired number of effector molecules may be separated from any starting materials or other product generated during the production process and containing an unwanted number of effector molecules by conventional means, for example by chromatography techniques such as ion exchange, size exclusion or hydrophobic interaction chromatography.

Also provided by the present invention is a mixture containing two or more antibody Fab or Fab′ fragments, characterized in that the mixture is enriched for Fab or Fab′ fragments in which the heavy chains in the fragments are not covalently bonded to the light chains, the fragments have two or more effector molecules attached and at least one of said molecules is attached to a cysteine in the light chain or the heavy chain constant region. Said mixture may be produced using the methods provided by the present invention. By ‘enriched’ we mean that the antibody fragment with the desired number of effector molecules attached accounts for 50% or greater of the mixture. Preferably the antibody fragment with the desired number of effector molecules attached accounts for between 50 and 99% of the mixture. Preferably the mixtures are enriched by greater than 50%, preferably greater than 60%, more preferably greater than 70%. The proportion of such mixtures containing the antibody fragment with the desired number of effector molecules may be determined by using the size exclusion HPLC methods described herein. In one example the mixture is enriched with a Fab′ fragment in which the heavy chain is not covalently bonded to the light chain and two or more effector molecules are attached to the fragment, wherein at least one effector molecule is attached to an interchain cysteine and at least one effector molecule is attached to the hinge region.

The antibody fragments according to the invention may be useful in the detection or treatment of a number of diseases or disorders. Such diseases or disorders may include those described under the general heading of infectious disease, e.g. bacterial infection; fungal infection; inflammatory disease/autoimmunity e.g. rheumatoid arthritis, osteoarthritis, inflammatory bowel disease; cancer; allergic/atopic disease e.g. asthma, eczema; congenital disease, e.g. cystic fibrosis, sickle cell anemia; dermatologic disease e.g. psoriasis; neurologic disease, e.g. multiple sclerosis; transplants e.g. organ transplant rejection, graft-versus-host disease; and metabolic/idiopathic disease e.g. diabetes.

The antibody fragments according to the invention may be formulated for use in therapy and/or diagnosis and according to a further aspect of the invention we provide a pharmaceutical composition comprising an antibody Fab or Fab′ fragment in which the heavy chain in the fragment is not covalently bonded to the light chain characterized in that two or more effector molecules are attached to the fragment and at least one of said molecules is attached to a cysteine in the light chain or the heavy chain constant region, together with one or more pharmaceutically acceptable excipients, diluents or carriers.

### EXAMPLES

The present invention will now be described by way of example only, in which reference is made to:

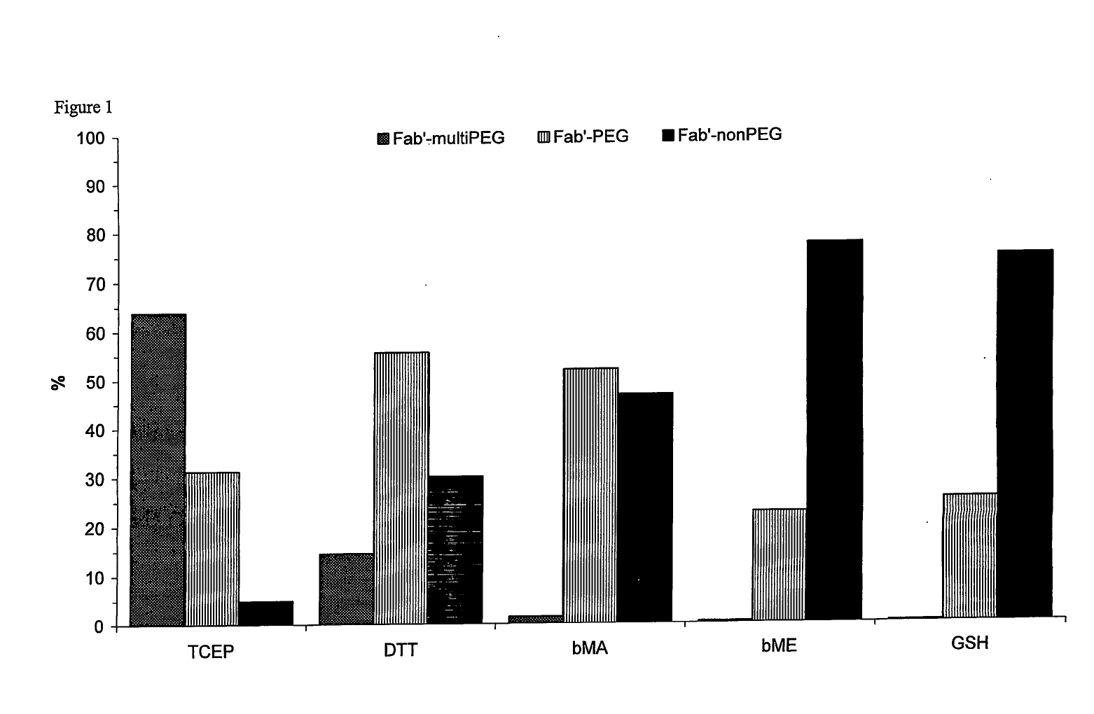

FIG. 1: Proportions of multi-PEGylated, mono-PEGylated and unPEGylated g165Fab′ LC-C HC-C, hinge-CAA produced using various reductants as determined by size exclusion HPLC.

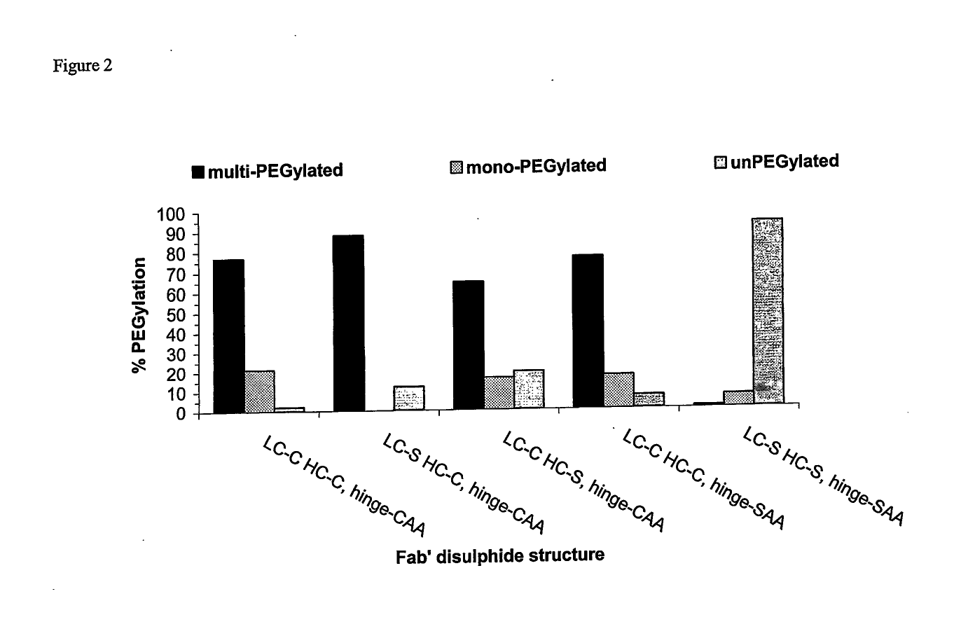

FIG. 2: Proportions of multi-PEGylated, mono-PEGylated and unPEGylated g165Fab′ variants produced using TCEP as the reductant, as determined by size exclusion HPLC.

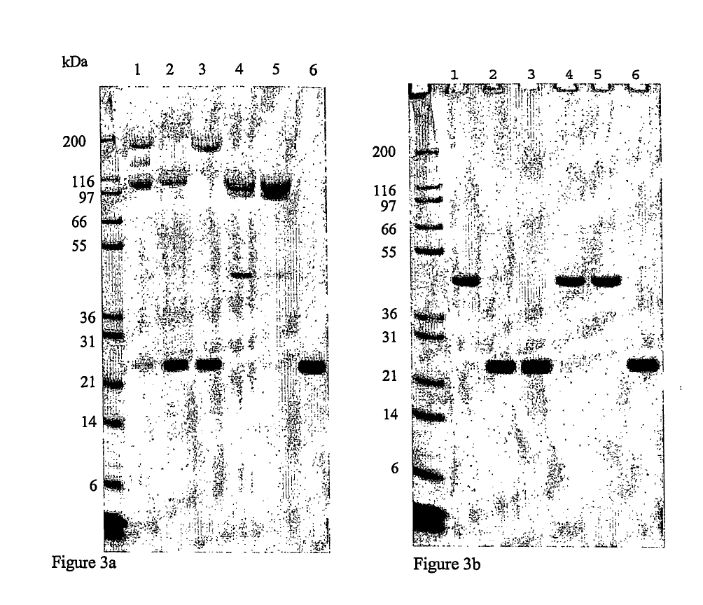

FIG. 3a: Non-reducing SDS-PAGE of PEGylated g165 Fab′ variants. Lane 1 Fab′ LC-C HC-C, hinge-CAA; Lane 3 Fab′ LC-S HC-C, hinge-CAA; Lane 4 Fab′ LC-C HC-S, hinge-CAA; Lane 5 Fab′ LC-C HC-C, hinge-SAA; Lane 6 Fab′ LC-S HC-S, hinge-SAA.

FIG. 3b: Non-reducing SDS-PAGE of purified g165 Fab′ variants. Lane 1 Fab′ LC-C HC-C, hinge-CAA; Lane 3 Fab′ LC-S HC-C, hinge-CAA; Lane 4 Fab′ LC-C HC-S, hinge-CAA; Lane 5 Fab′ LC-C HC-C, hinge-SAA; Lane 6 Fab′ LC-S HC-S, hinge-SAA.

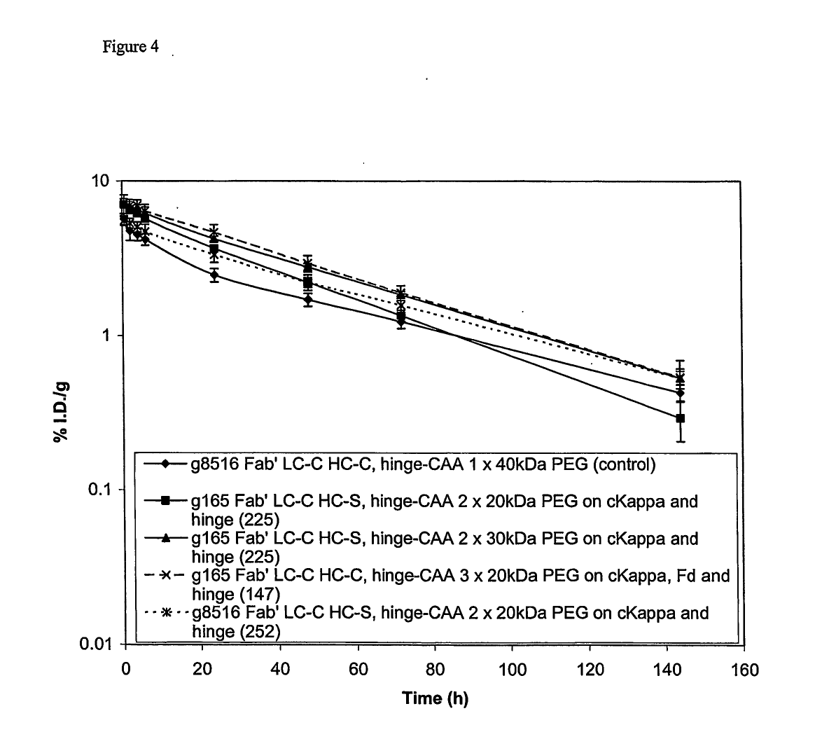

FIG. 4: Pharmacolinetics of intravenously dosed ¹²⁵I labelled PEGylated Fab′ in rats

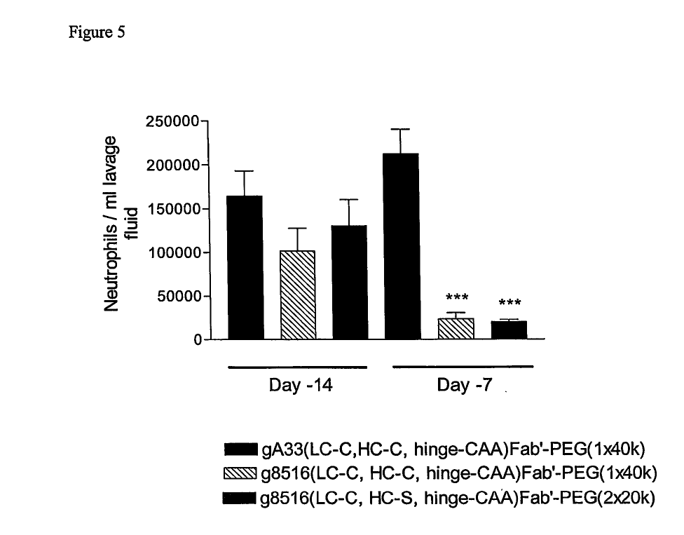

FIG. 5: Neutralisation of intraperitoneal dosed antigen-induced neutrophil accumulation by intravenous pre-dosing of Fab′-PEG in mice. ***p<0.001 compared to control antibody.

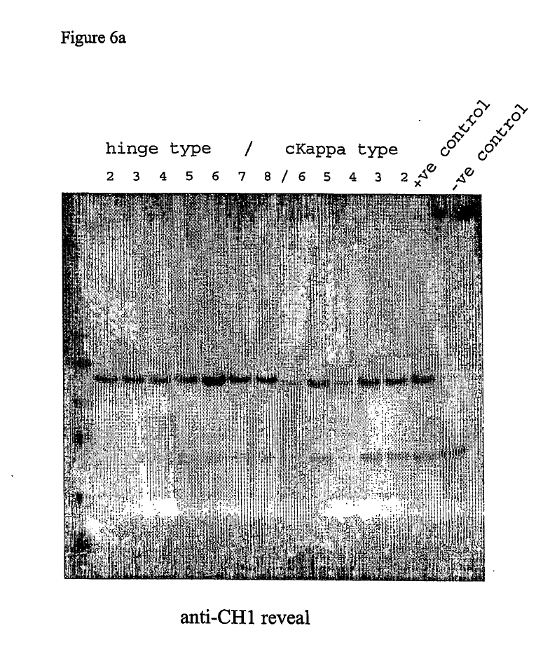

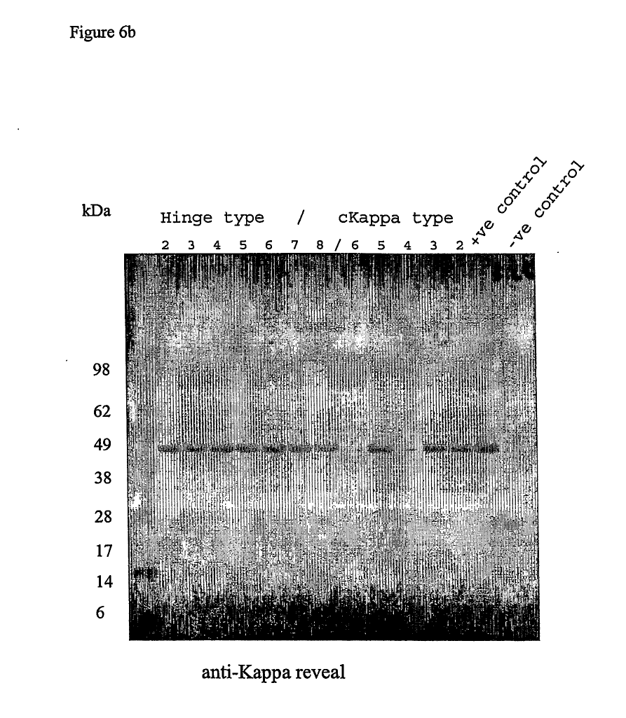

FIGS. 6a and 6b: Non-reducing SDS-PAGE immunoblotting of hinge and light chain pairs to illustrate disulphide bonding between the light chain and the hinge.

FIG. 7: Hinge and light chain sequences

### FAB′ NOMENCLATURE AND GENERAL METHODS

The Fab and Fab′ molecules used in the following examples are g165 which binds to a human cell surface receptor and g8516 which binds to the human cytokine IL-1β. The nomenclature for each fragment uses the single letter code C for cysteine and S for serine to denote the amino acid at the site of the inter-chain cysteine of C_{L} in the light chain (LC) and the site of the inter-chain cysteine of C_{H}1 in the heavy chain (HC). For example, a normal Fab′ is ‘g165 Fab′ LC-C HC-C, hinge-CAA’ whereas the version in which the inter-chain cysteine of C_{H}1 has been substituted with a serine so there is no inter-chain disulphide is eg. ‘g165 Fab′ LC-C HC-S, hinge-CAA’. A full γ1 middle hinge is noted as ‘hinge-CPPCPA’. A list of the plasmids used in the following examples are shown in Table 1.

TABLE 1
Plasmid and protein details.

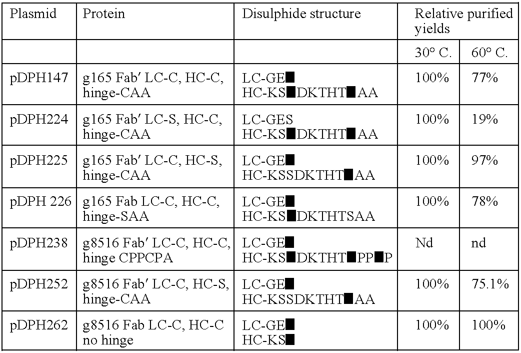

Production of Fab′

Fab′ molecules of the present invention were produced in E. coli strain W3110 and purified using standard methods (Humphreys et al., 2002, Protein Expression and Purification, 26, 309-320). PCR mutagenesis was used to change the interchain cysteines of C_{L} and C_{H}1 to serines.

Reduction and PEGylation of Fab′.

All reductions and PEGylations were performed in 0.1M Phosphate pH6.0; 2 mM EDTA. The concentration of Fab′ and reductant were as stated in each example. In all cases reduction was done for 30 minutes at room temperature (˜24° C.), the proteins desalted on a PD-10 column (Pharmacia) and then mixed with 5 fold molar excess of PEG-maleimide over Fab′. The 40 kDa PEG was from Nektar and 20 and 30 kDa PEG was from Nippon Oils and Fats (NOF). PEGylated Fab′ was separated from unpegylated Fab′ by size exclusion HPLC on analytical Zorbax GF-450 and GF-250 columns in series. These were developed with a 30 min isocratic gradient of 0.2M phosphate pH 7.0+10% ethanol at 1 ml/min and Fab′ detected using absorbance at 214 nm and 280 nm.

### Example 1

### Creation of Novel PEGylated Fab′ Fragments

A tri-PEGylated antibody Fab′ fragment was produced by reducing the inter-chain disulphide of the antibody fragment g165 Fab′ LC-C HC-C, hinge-CAA and attaching PEG molecules to the available thiols of the inter-chain cysteines of C_{L} and C_{H}1 and the hinge cysteine. A number of different reductants were tested. The thiol based reductants reduced glutathione (GSH), β-mercaptoethanol (β-ME), β-mercaptoethylamine (β-MA) and dithiothreitol (DTT) and the non-thiol based reductant tris carboxyethyl phosphine (TCEP).

The g165 Fab′ LC-C HC-C, hinge-CAA was at 10 mg/ml and the reductants were at 5 mM and the number of PEG molecules attached to the fragments was determined by size exclusion HPLC (FIG. 1). PEGylation was expected to occur on all three available cysteines if the inter-chain disulphide was reduced. TCEP resulted in 65% multi-PEGylation whilst DTT only resulted in approximately 15% multi-PEGylated material and β-MA, β-ME and GSH only resulted in trace amounts (<1%) of multi-PEGylation. The thiol based reductants typically resulted in monoPEGylated Fab′ as these reductants were not strong enough to reduce the inter-chain disulphide bond. These are the reductants typically used in the production of PEGylated antibody fragments where the interchain disulphide is retained. The low efficiency of mono PEGylation achieved using these reductants was observed here, 55% for DTT, 52% βMA, 20% βME and 22% GSH.

In another example, the inter-chain disulphide linkage between the heavy and the light chain was removed by replacing either the interchain cysteine of C_{L} or the interchain cysteine of C_{H}1 with serine. Each antibody fragment at 10 mg/ml was reduced with 5 mM TCEP, desalted and then reacted with 40 kDa PEG-maleimide. The results in FIGS. 2 and 3 show that all of the cysteines were highly accessible to the PEG maleimide. In all cases the predicted number of thiols (2 or 3) were accessible after reduction with TCEP allowing efficient site specific PEGylation to occur. FIG. 3b shows the unPEGylated purified Fab′ fragments. FIG. 3a illustrates the increase in molecular weight associated with the attachment of two or more PEG molecules. Lane 1 corresponds to LC-C HC-C, hinge CAA where two PEG molecules are attached to the heavy chain and one to the light chain. The highest molecular weight band in lane 1 is the heavy chain with two PEG molecules attached, the next band is a small amount of the heavy chain with only one PEG molecule attached and the next band is the light chain with only one PEG molecule attached. Lane 3 corresponds to Fab′ LC-S HC-C, hinge CAA in which there are two PEG molecules attached to the heavy chain. The highest molecular weight band in lane 3 is the heavy chain with two PEG molecules attached while the lower molecular weight band is free light chain with no PEG molecules attached. Lane 4 corresponds to Fab′ LC-C HC-S, hinge CAA in which there is one PEG on the heavy and the light chain. The two high molecular weight bands very close together are heavy and light chain with one PEG molecule attached. The lower band is a small amount of presumed covalent light chain dimer with no PEG attached. Lane 5 is the same as lane 4 in that a single PEG is attached to each chain of Fab′ LC-C HC-C, hinge SAA. Lane 6 is the control in which there is no interchain disulphide and no PEG molecules attached, Fab′ LC-S HC-S, hinge SAA. The one major band observed is that of non-covalently associated heavy and light chains.

In all cases >65% of Fab′ molecules were multi PEGylated with either 2 or 3 PEG molecules. The modified antibody fragments of the present invention can therefore be produced more efficiently than conventional antibody fragments where the interchain disulphide is retained.

The non-thiol based reductant tris carboxyethyl phosphine (TCEP) was shown to be a more efficient reducing agent than the thiol based reductants reduced glutathione (GSH), β-mercaptoethanol (β-ME), β-mercaptoethylamine (β-MA) and dithiothreitol (DTT). TCEP is therefore a useful reducing agent for producing the modified antibody fragments of the present invention.

### Example 2

### Stability tests of Fab′ lacking inter CL:C_{H}1 disulphide

Effect of Lack of Inter CL:C_{H}1 Disulphide Bonds on the Physical Performance of Fab′ and Fab-PEG.

i) Purification of Fab′

Antibody fragments produced in E. coli are usually extracted from the periplasm by shaking overnight in Tris/EDTA at 30° C. or 60° C. The high temperature heat extraction facilitates the extraction and partial purification from E. coli proteins of antibody fragments (see U.S. Pat. No. 5,665,866). We observed that yields of Fab′ in which the light chain cysteine had been substituted for serine were reduced in the order of 80% when the incubation was done at 60° C. relative to that of 30° C. (Table 1). Suprisingly, where the heavy chain cysteine was substituted for serine stability was greater than 95% at 60° C. which indicated that the Fab′ LC-C HC-S, hinge-CAA had a long and flexible enough hinge to efficiently form a disulphide between C_{L} and the hinge, making this a useful intermediate in the production of diPEGylated Fab′ molecules as this can be purified using the heat extractions described above. Non reducing SDS-PAGE of such Fab′ (Lane 4, FIG. 3b) also demonstrate a covalent linkage between LC and HC. FIG. 3b shows that in lane 3, LC-S HC-C, hinge CAA is present as free heavy and light chain whereas in lane 4 LC-C HC-S, hinge CAA the heavy and light chains are covalently linked, giving this Fab′ the same migration as a Fab′ in which the native interchain disulphide is present e.g. lane 1, Fab′ LC-C HC-C, hinge CAA.

Fab′ engineered to lack inter C_{L}:C_{H}1 disulphide bonds were purified using protein G or ion exchange in exactly the same manner as Fab′ containing inter C_{L}:C_{H}1 disulphide bonds. Since these involved elution at pH 2.7 (protein G) or equilibration at pH 4.5 (ion exchange) the Fab′ interaction between C_{L}:C_{H}1 was clearly physico-chemically stable.

ii) Antigen Binding Affinity In Vitro.

g165 Fab′ with PEG molecules attached in the presence or absence of a covalent linkage between the light chain and the heavy chain were analysed for antigen affinity using BIAcore™. Antigen was captured on a BIAcore™ chip and the antibodies passed over in the solution phase and an affinity determined.

TABLE 2
Antigen affinity of mono, di- and tri- PEGylated Fab′ in vitro.

| | | ka e5 | kd e-4 | KD |
|---|---|---|---|---|
| SAMPLE | Fab′ | (1/Ms) | (1/s) | nM |
| 147 | g165 LC-C | 6.6 | 8.5 | 1.3 |
| | HC-C, | | | |
| | hinge-CAA | | | |
| 224 | g165 LC-S | 6.6 | 10.5 | 1.6 |
| | HC-C, | | | |
| | hinge-CAA | | | |
| 225 | g165 LC-C | 6.7 | 8.5 | 1.3 |
| | HC-S, | | | |
| | hinge-CAA | | | |
| 226 | g165 LC-C | 5.3 | 7.6 | 1.4 |
| | HC-C, | | | |
| | hinge-SAA | | | |
| 147 1 × | g165 LC-C | 6.5 | 11.9 | 1.8 |
| 40 PEG | HC-C, | | | |
| | hinge-CAA | | | |
| 147 3 × | g165 LC-C | 6.8 | 13.3 | 1.9 |
| 20 PEG | HC-C, | | | |
| | hinge-CAA | | | |
| 225 2 × | g165 LC-C | 8.2 | 11.9 | 1.4 |
| 20 PEG | HC-S, | | | |
| | hinge-CAA | | | |
| 225 2 × | g165 LC-C | 8.1 | 13.4 | 1.6 |
| 30 PEG | HC-S, | | | |
| | hinge-CAA | | | |

Table 2 shows that neither the lack of inter C_{L}:C_{H}1 disulphide or presence of mono- di- or tri-PEGylation materially affects the binding affinity.

### Example 3

### Pharmacokinetics of Fab-PEG in Rats

Circulating Half Life of Fab PEGylated on Both Polypeptides in Animals.

¹²⁵I labelled PEGylated Fab′ molecules were injected intravenously into rats and the serum permanence of potential therapeutic Fab′ determined. The circulating half life of non-PEGylated Fab′ is very short (t½β≈30 minutes) and that of free LC or HC is likely to be shorter still.

300 μg of Fab′-PEG per animal group was ¹²⁵I-labelled using Bolton and Hunter reagent (Amersham) to a specific activity of 0.22-0.33 μCi/μg.

Male Sprague Dawley rats of 220-250 g (Harlan) were injected intra veniously or sub cutaneously with 20 μg ¹²⁵I-labelled Fab′-PEG variants whilst under Halothane anaesthesia (n=6 per group). Serial arterial bleeds from the tail were taken at 0.5, 2, 4, 6, 24, 48, 72 and 144 hours post administration. Samples were counted using a COBRA™ Autogamma counter (Canberra Packard). Data were plotted and Area Under Curve were calculated using GraphPad Prism (GraphPad Software Incorporated) and is expressed as % injected dose hour (% i.d/hr). The t½a is defined by time points 0.5, 2, 4, and 6, whilst the t½™ is defined by time points 24, 48, 72 and 144.

To test whether the non-covalent association between C_{L} and C_{H}1 would be disturbed by the steric issues relating to the maleimide linker and PEG, g165 Fab′ LC-C HC-S, hinge-CAA was di-PEGylated with both 20 and 30 kDa PEG using TCEP as the strong reducing agent. In addition, a normal g165 Fab′ LC-C HC-C, hinge-CAA was tri-PEGylated with 20 kDa PEG by virtue of a very strong reduction with TCEP. The data in Table 2 and FIG. 4 show that although the final PEGylated forms of these Fab′ have non-covalently associated LC and HC the circulating half life is comparable to that of a mono-PEGylated control.

TABLE 3
Pharmacokinetic analysis of Fab-PEG in rat model.

| Fab | PEG | Admin. | t½ α (h) | T½ β (h) | AUC (0-∞) (% dose*h) |
|---|---|---|---|---|---|
| g8516 Fab′ LC-C HC-C Hinge-CAA | 1 × 40 kDa (branched) | i.v. | 4.76 ± 1.3 | 48 ± 2.8 | 4554 ± 268 |
| g165 Fab′ LC-C HC-S hinge-CAA | 2 × 20 kDa | i.v. | — | 31 ± 2.8 | 4786 ± 353 |
| g165 Fab′ LC-C HC-S hinge-CAA | 2 × 30 kDa | i.v. | — | 39 ± 2.0 | 6154 ± 369 |
| g165 Fab′ LC-C HC-C hinge-CAA | 3 × 20 kDa | i.v. | — | 38 ± 1.1 | 6171 ± 693 |

### Example 4

### Mouse Antigen Binding Efficacy Models: In Vivo Efficacy in Animal Models.

i.v. Dosed g8516 Fab′-PEG and Intraperitoneal Dosed hIL-1β.

Male Balb/c mice (21 g) were injected intravenously (i.v.) with a single dose (3 mg/kg in 100 μPBS) of g8516 Fab′LC-C HC-C hinge-CAA-40 kDa PEG, g8516 Fab′LC-C HC-S hinge-CAA-2×20 kDa PEG, or ghA33 Fab′LC-C HC-C hinge-CAA-40 kDa PEG (irrelevant control), 7 and 14 days prior to an ip. injection of hIL-1β (3 ng/kg in 100 μl PBS vehicle). After 120 minutes, mice were killed by cervical dislocation and peritoneal lavage was performed (3 ml PBS+0.25% BSA, 12 mM HEPES). A total leukocyte count was performed using a Coulter Counter. For identification of neutrophils, 50 μl peritoneal lavage fluid was stained with 1:300 dilution of anti-CD45-CyChrome mAb and 1:300 dilution of anti-GR-1-PE mAb (anti-Ly6G/Ly6C) for 20 minutes (4° C., in the dark). Leukocytes were washed once in PBS (0.25% BSA, 12 mM HEPES), resuspended in 300 μl PBS (0.25% BSA, 12 mM HEPES) and analysed by flow cytometry. Neutrophils were identified as CD45⁺GR-1^{HIGH}.

FIG. 5 shows that there was no difference between g8516 Fab-PEG that have, or lack inter C_{L}:C_{H}1 disulphide bonds at either of the time points. This demonstrates that efficacy is retained during 1 week in the mouse circulation and therefore by implication that LC and HC remain associated during this time.

### Example 5

### Design and Testing of Hinge Sequences and Modified Light Chain Sequences

Following the observations made in Example 2 constructs were made and tested to investigate the limits of flexibility for forming an interchain disulphide between a light chain cysteine of cKappa and the hinge cysteine of an antibody Fab′ fragment in which the interchain cysteine of C_{H}1 was replaced with serine. Various constructs were made containing 7 different hinge sequences (SEQ ID NOs 5-11) and tested for their ability to form interchain disulphide bond between the LC and Hinge during E. coli expression, periplasmic extraction at 60° C. and non-reducing SDS-PAGE and immunoblotting. All hinge variants were combined with a standard cKappa from IgG1 (SEQ ID NO: 15). We found that all variants made (both stiffer, more flexible and longer) are able to form a disulphide bond to cKappa. (FIGS. 6a and 6b, lanes 2-8 (SEQ ID NOs 5-11 respectively)). The positive control was a Fab′ containing an interchain disulphide bond. The negative control was a Fab′ lacking an interchain disulphide bond (both interchain cysteines having been substituted with serine).

Also tested were alternative cysteine positions in cKappa in an antibody Fab′ fragment in which both the interchain cysteine of C_{L} and C_{H}1 were replaced by serine. The terminal Cys that normally forms the interchain disulphide was mutated to Ser whilst the Cys was moved one amino acid at a time toward the N-terminus. Five different ckappa sequences were tested (SEQ ID NOs 16-20). These were paired with a hinge (SEQ ID NO:2) known to be capable of forming a disulphide linkage with the interchain cysteine of the light chain at position 214 to test whether the linkage was still formed when the cysteine of ckappa was in a different position. We found that all variants made were able to form a disulphide bond to the hinge, as determined by Non-reducing SDS-PAGE and immunoblotting. (FIGS. 6a and 6b, lanes 9-13 cKappa type 6-2 (SEQ ID NOs 20, 19, 18, 17 and 16 respectively). The positive control was a Fab′ containing an interchain disulphide bond. The negative control was a Fab′ lacking an interchain disulphide bond (both interchain cysteines having been substituted with serine).

From the above examples it can clearly be seen that the novel PEGylated molecules of the present invention can be produced more efficiently than PEGylated antibodies that contain an inter C_{L}:C_{H}1 disulphide bond. The examples also demonstrate that PEGylation of Fab′ which lack the interchain disulphide bond has no adverse effects on the biological activity or stability of the antibody Fab′ making these useful therapeutic molecules which can be produced more efficiently than conventional Fab′.

1. An antibody fragment comprising a Fab or Fab′ fragment; that has been modified by replacement of either the interchain cysteine of C_{H}1 or the interchain cysteine of C_{L} with another amino acid.

2. The antibody fragment of claim 44 comprising a modified hinge region.

3. The antibody fragment of claim 2 wherein the hinge region comprises any one of the sequences provided in SEQ ID Nos 1-14.

4. The antibody fragment of claim 2 wherein the C_{L} interchain cysteine is covalently bonded to a cysteine in the hinge region.

5. An antibody Fab′ fragment in which both the interchain cysteine of C_{H}1 and the interchain cysteine of C_{L} have been replaced by another amino acid and an engineered cysteine in the light chain constant region is covalently bonded to a cysteine in the hinge region.

6. The antibody fragment of claim 5 wherein the light chain constant region comprises any one of the sequences provided in SEQ ID Nos 16-20.

7. The antibody fragment of claim 6 wherein the hinge region comprises any one of the sequences provided in SEQ ID Nos 1-11.

8. The antibody fragment of claim 1 wherein the fragment is a Fab′ fragment in which the interchain cysteine of C_{L} has been replaced by another amino acid.

9. The antibody fragment of claim 8 wherein the fragment contains a modified hinge region.

10. The antibody fragment of claim 1 wherein the fragment is a Fab fragment in which the C_{H}1 interchain cysteine has been replaced by another amino acid.

11. The antibody fragment of claim 1 wherein the fragment is a Fab fragment in which the C_{L} interchain cysteine has been replaced by another amino acid.

12. The antibody fragment of claim 1 wherein the interchain cysteine that has been replaced has been replaced by a non-thiol containing amino acid.

13. The antibody fragment of claim 12 wherein the non-thiol containing amino acid is serine.

14. The antibody fragment of claim 1 wherein at least two effector molecules are attached to the fragment.

15. The antibody fragment of claim 14 wherein an effector molecule is attached to a cysteine in the light chain constant region and/or to a cysteine in the heavy chain constant region.

16. The antibody fragment of claim 15, wherein an effector molecule is attached to a cysteine in the light chain constant region and to a cysteine in the heavy chain constant region, wherein the two cysteines would otherwise be linked to each other via a disulphide bond if the effector molecules were not attached.

17. The antibody fragment of claim 14 wherein an effector molecule is attached to the interchain cysteine of C_{L}, to the interchain cysteine of C_{H}1, or to an engineered cysteine in the light chain constant region.

18. The antibody fragment of claim 1 wherein the fragment is a Fab′ fragment in which an effector molecule is attached to each cysteine in the hinge region.

19. The antibody fragment of claim 18 wherein an effector molecule is attached to a cysteine in the hinge region that was covalently linked to the interchain cysteine of C_{L} prior to attachment of the effector molecules.

20. The antibody fragment of claim 18 wherein an effector molecule is attached to a cysteine in the hinge region that was covalently linked to an engineered cysteine in the light chain constant region prior to attachment of the effector molecules.

21. A method of producing an antibody fragment of claim 14 comprising:
a. treating an antibody Fab or Fab′ fragment in which either the interchain cysteine of C_{H}1 or the interchain cysteine of C_{L} has been replaced by another amino acid with a reducing agent capable of generating a free thiol group in at least one cysteine of the heavy and/or light chain constant region and/or, where present, the hinge; and
b. reacting the treated fragment with an effector molecule.

22. The method of claim 21 wherein step (a) further comprises reducing the covalent bond between the C_{L} interchain cysteine and a cysteine in the hinge region.

23. The method of claim 21 wherein step (a) further comprises reducing the covalent bond between an engineered cysteine in the light chain constant region and a cysteine in the hinge region.

24. An antibody fragment comprising a Fab or Fab′ fragment that has been modified by attachment of two or more effector molecules wherein the heavy chain in the fragment is not covalently bonded to the light chain, and an effector molecule is attached to each of the interchain cysteines of C_{L} and C_{H}1.

25. The antibody fragment of claim 24 wherein at least one further effector molecule is attached to a cysteine in the light chain constant region and/or to a cysteine in the heavy chain constant region.

26. The antibody fragment of claim 25, wherein an effector molecule is attached to a cysteine in the light chain constant region and to a cysteine in the heavy chain constant region, and the two cysteines would otherwise be linked to each other via a disulphide bond if the effector molecules were not attached.

27. The antibody fragment of claim 26 wherein the fragment is a Fab′ fragment that contains a modified hinge region.

28. The antibody fragment of claim 27 wherein the hinge region comprises any one of the sequences provided in SEQ ID Nos 1-14.

29. The antibody fragment of claim 24 wherein the fragment is a Fab′ fragment and an effector molecule is attached to at least one cysteine in the hinge region.

30. A method of producing an antibody fragment of claim 24 comprising:
a. treating an antibody Fab or Fab′ fragment with a reducing agent capable of generating a free thiol group in at least the interchain cysteine of C_{H}1 and the interchain cysteine of C_{L}; and
b. reacting the treated fragment with an effector molecule.

31. The antibody fragment of claims 1 or 24 wherein the interchain cysteine of C_{L} is at position 214 of the light chain and the interchain cysteine of C_{H}1 is at position 233 of the heavy chain.

32. The method of claims 21 or 30 wherein the reducing agent is a non-thiol based reducing agent.

33. The method of claim 32 wherein the reducing agent is a trialkylphosphine.

34. The method of claim 33 wherein the trialkylphosphine reducing agent is tris(2-carboxyethyl)phosphine (TCEP).

35. The method of claim 33 wherein the trialkylphosphine reducing agent is tris(3-hydroxypropyl)phosphine (THP).

36. The method of claim 21 wherein either or both of steps (a) and (b) are performed in the presence of a chelating agent.

37. The method of claim 36 wherein the chelating agent is EDTA.

38. The method of claim 37 wherein both steps (a) and (b) are performed in the presence of EDTA.

39. A composition comprising a mixture of two or more antibody Fab or Fab′ fragments, wherein the mixture is enriched for Fab or Fab′ fragments in which the heavy chains in the fragments are not covalently bonded to the light chains, the fragments have two or more effector molecules attached, and at least one of said effector molecules is attached to a cysteine in the light chain or the heavy chain constant region of the fragments.

40. The composition of claim 39 wherein greater than 50% of the mixture comprises Fab′ or Fab fragments in which the heavy chains in the fragments are not covalently bonded to the light chains, the fragments have two or more effector molecules attached, and at least one of said effector molecules is attached to a cysteine in the light chain or the heavy chain constant region of the fragments.

41. The antibody fragment of claims 14 or 24 wherein the effector molecule is PEG.

42. A host cell expressing the antibody fragment of claim 1.

43. A pharmaceutical composition comprising an antibody fragment of claims 1 or 24, together with one or more pharmaceutically acceptable excipients, diluents or carriers.

44. The antibody fragment of claim 1 wherein the fragment is a Fab′ fragment in which the interchain cysteine of C_{H}1 has been replaced by another amino acid.


SEQUENCE LISTING (20 sequences)

SEQ ID NO: 1 - protein, 8 aa, Artificial Sequence
  Synthetic Construct
DKTHTCPP

SEQ ID NO: 2 - protein, 8 aa, Artificial Sequence
  Synthetic Construct
DKTHTCAA

SEQ ID NO: 3 - protein, 11 aa, Artificial Sequence
  Synthetic Construct
DKTHTCPPCPA

SEQ ID NO: 4 - protein, 11 aa, Artificial Sequence
  Synthetic Construct
DKTHTCAACPA

SEQ ID NO: 5 - protein, 8 aa, Artificial Sequence
  Synthetic Construct
GGGGGCAA

SEQ ID NO: 6 - protein, 7 aa, Artificial Sequence
  Synthetic Construct
DKPTCAA

SEQ ID NO: 7 - protein, 8 aa, Artificial Sequence
  Synthetic Construct
DKPPTCAA

SEQ ID NO: 8 - protein, 8 aa, Artificial Sequence
  Synthetic Construct
DPPPTCAA

SEQ ID NO: 9 - protein, 9 aa, Artificial Sequence
  Synthetic Construct
SDKTHTCAA

SEQ ID NO: 10 - protein, 10 aa, Artificial Sequence
  Synthetic Construct
SSDKTHTCAA

SEQ ID NO: 11 - protein, 11 aa, Artificial Sequence
  Synthetic Construct
SSSDKTHTCAA

SEQ ID NO: 12 - protein, 4 aa, Artificial Sequence
  Synthetic Construct
GCAA

SEQ ID NO: 13 - protein, 8 aa, Artificial Sequence
  Synthetic Construct
GKTHTCAA

SEQ ID NO: 14 - protein, 9 aa, Artificial Sequence
  Synthetic Construct
GDKTHTCAA

SEQ ID NO: 15 - protein, 10 aa, Artificial Sequence
  Synthetic Construct
VTKSFNRGEC

SEQ ID NO: 16 - protein, 10 aa, Artificial Sequence
  Synthetic Construct
VTKSFNRGCS

SEQ ID NO: 17 - protein, 10 aa, Artificial Sequence
  Synthetic Construct
VTKSFNRCES

SEQ ID NO: 18 - protein, 10 aa, Artificial Sequence
  Synthetic Construct
VTKSFNCGES

SEQ ID NO: 19 - protein, 10 aa, Artificial Sequence
  Synthetic Construct
VTKSFCRGES

SEQ ID NO: 20 - protein, 10 aa, Artificial Sequence
  Synthetic Construct
VTKSCNRGES